(19) United States
(12) Patent Application Publication
(10) Pub. No.: US 2013/0186935 A1
(43) Pub. Date: Jul. 25, 2013

(54) VASCULAR ANASTOMOTIC STAPLERS

(71) Applicants: John K. Edoga, NorthBeach, NJ (US); Thierry Richard, Florham Park, NJ (US)

(72) Inventors: John K. Edoga, NorthBeach, NJ (US); Thierry Richard, Florham Park, NJ (US)

(73) Assignee: EDRICH HEALTH TECHNOLOGIES, INC., Morristown, NJ (US)

(21) Appl. No.: 13/556,171

(22) Filed: Jul. 23, 2012

Related U.S. Application Data
(63) Continuation of application No. 12/293,932, filed on Mar. 25, 2009, now abandoned.
(60) Provisional application No. 60/786,332, filed on Mar. 27, 2006.
(60) Provisional application No. 60/784,981, filed on Mar. 23, 2006.

(51) Int. Cl. A61B 17/068

(52) U.S. Cl. CPC A61B 17/068
USPC 227/178.1

(57) ABSTRACT

Vascular anastomotic staplers (100, 200, 300) for securing grafts to vessels and methods of using such staplers are disclosed. The staplers are capable of performing anastomoses between relatively small vessels (184, 240, 340) and grafts (186, 236, 316) such as prosthetic vascular grafts. The staplers are capable of discharging multiple staples simultaneously suitable for end-to-end anastomosis (200) and for end-to-side anastomosis (300).

### CROSS-REFERENCE TO RELATED APPLICATIONS

This application claims the benefit of priority from and is a continuation of U.S. patent application Ser. No. 12/293.932, filed Mar. 25. 2009, which in turn claims the benefit of priority to and is a National Stage Entry of International (PCT) Application No. PCT/US07/07364, which in turn claims the benefit of priority of U.S. Provisional Patent Application Ser. No. 60/786.332 filed Mar. 27, 2006 and U.S. Provisional Patent Application Ser. No. 60/784.981 filed Mar. 23, 2006. The disclosure of each of the aforementioned patent applications is incorporated by reference herein in its entirety for any purpose whatsoever.

### BACKGROUND OF THE INVENTION

It is well known that grafts may be affixed to vessels in a human body during numerous medical procedures. For example, U.S. patent application Ser. No. 10/837,827, and PCT Application No. PCT/US06/44653, which are incorporated by reference herein in their entirety and assigned to the applicant, disclose staplers for securing a graft to a vessel. The staplers include a staple housing for storing staples and a staple exit area associated with each of the staples for discharging staples therethrough. The staplers also include an actuating assembly adapted for discharging the staples through the staple exit areas, and a displacement mechanism operative for pushing the staple exit areas against the graft when discharging the staples therethrough.

Also well known are staplers for use in fixation of grafts to the walls of vessels in an end-to-side anastomosis as well as in an end-to-end anastomoses during conventional vascular surgery. For example, U.S. patent application Ser. No. 10/737,630, which is incorporated by reference herein in its entirety and is assigned to the applicant, discloses staplers for securing a graft or prosthesis to a vessel in an end-to-side or in an end-to-end anastomosis. The staplers include a staple housing for storing staples adapted to be discharged through corresponding staple exit areas. The stapler discharges staples in a manner to secure a graft, a bolster and a vessel together to form end-to-side anastomosis as well as end-to-end anastomosis.

### SUMMARY OF THE INVENTION

The present application discloses improved vascular anastomotic staplers for securing grafts to vessels and methods of using such staplers. The staplers are capable of performing anastomoses between relatively small vessels and grafts such as prosthetic vascular grafts. In one embodiment, the stapler is adapted to perform end-to-end anastomoses and deploy multiple staples simultaneously. The stapler employs W-shaped staples arranged in a circumference at the distal end of the stapler adjacent corresponding pushers and an actuator with a detent. The staples can be made of titanium, stainless steel, memory alloys or other similar material. In operation, the actuator is advanced or displaced forward toward the distal end of the stapler which causes the pushers and staples to be displaced laterally with the staples being pushed out through the staple exit sites while the detente hold unto the central portion of the staples. This causes the staples to close into B-shaped forms as they exit the stapler. Second, as forward displacement of the actuator continues to move forward, protuberances on sides of the actuator engage arms of the detent and cause the detent to be simultaneously rotated laterally thereby releasing the staples to engage a vessel and graft.

In another embodiment, the stapler is capable of discharging multiple staples simultaneously and which is suitable for end-to-end anastomosis and for end-to-side anastomosis. The stapler includes memory alloy staples capable of exiting through a distal end of the stapler, bolsters, and a graft. The assembly is configured such that its lead end can be inserted into a cavity of a vessel through an arteriotomy. For end-to-end anastomosis, the staples are displaced forward by pushers, the staples return to their natural shape and at the same time incorporate the graft, the vessel and bolster to provide a water tight anastomosis.

The stapler can be adapted for end-to-side anastomosis. The stapler includes a graft loaded on a central shaft (with a split tip) and between the central shaft and the staple cartridge. The distal end of the graft curls around the distal end of the cartridge to cover a corresponding staple exit area. As a result, when the staples are pushed out through the exit areas, the staples penetrate the graft from outside to inside, and then as the staples attempt to return to their natural shape, engage the vessel inside wall and pierce the vessel inside wall to the vessel outside wall to then engage the bolster on the anvil. In this manner, the staplers provide a water tight anastomosis.

In one aspect of a first embodiment of the present application, disclosed is a stapler for simultaneously discharging a series of staples. The stapler includes an outer housing and an actuator with a plurality of pushers configured within the outer housing such that advancement along the outer housing of the actuator forces the pushers radially outward toward the outer housing. The stapler includes a plurality of staples, each of the staples retained within one of the plurality of pushers. The stapler includes a staple detent having a plurality of arms, each of the arms is adapted for securing one of the plurality of staples against one of the pushers. The stapler also includes a plurality of staple exit areas formed through the outer housing, wherein the actuator may be advanced to force the pushers radially outward to discharge the staples through the staple exit areas.

In one or more of the embodiments of the first aspect of the present invention, the stapler discharges staples adapted to penetrate a vessel wall and a graft wall and secure the graft to the vessel. The stapler may be capable of discharging the staples through the staple exit areas simultaneously. The staples may be made from a memory alloy such as Nitinol or non memory alloys such as, titanium, stainless steel or the like. The staples may have a generally W-shape while stored in the stapler and a generally B-shape when discharged from the stapler. The staple may be formed from a plurality of U-shaped sections connected by a central element. The stapler may be formed from a plurality of opposed loops connected by a central element. The staple may have a first condition in which the opposed loops are open while stored in the stapler and a second condition in which the opposed loops are at least partially closed when discharged from an associated staple exit area. The staples may be disposed in a circle about a circumference within the outer housing. The staple detent may comprise a central hub connecting the plurality of arms thereto. The staple detent is adapted to rotate about a longitudinal axis within the outer housing. The staple detent is adapted to rotate between a first position to retain the staples within the outer housing and a second position to release the staples from the staple exit areas. The stapler may include a graft attached to the outer housing.

In another aspect of the first embodiment of the present application, disclosed is a method of attaching a graft to a vessel using a stapler of the first embodiment. The method includes providing a stapler with an outer housing at a distal end of the stapler comprising: a graft disposed upon the outer housing; a staple detent with a plurality of arms, each of the arms retaining one of a plurality of staples, the detent adapted to release the staples from an associated staple exit area; and a staple actuating mechanism to discharge the staples through an associated staple exit area by displacing the pushers laterally. The distal end of the stapler is then inserted within the vessel such that the staple exit areas and the graft are adjacent to the inside wall of the vessel at the site selected for the anastomosis. The actuating mechanism is then actuated to discharge the staples from the staple exit areas and to cause the staples to penetrate the graft and the vessel. The detents are actuated by the continued forward movement of the actuators to release the discharged staples from the staple exit areas.

In one or more of the embodiments of the above method, the step of inserting the distal end of the stapler within the vessel may include positioning a wall of the graft adjacent to a wall of the vessel. The actuating step may include advancing an actuator in a longitudinal direction which causes a pusher associated with a staple to move radially outward from a center of the stapler thereby discharging the staples. Actuating the detent to release the discharged staples may include rotating the detent about a central axis of the stapler thereby causing the arms to release the staples through the staple exit areas. The staples may be discharged through the staple exit areas simultaneously.

In an aspect of a second embodiment of the present application, disclosed is a surgical stapler system that includes a casing with a plurality of staple exit areas; a cartridge within the casing, the cartridge includes a plurality of radially mounted pushers with distal ends; a plurality of staples, one of the plurality of staples is associated with each of the pushers; an actuating mechanism adapted to advance the pushers along the cartridge to push the staples through the staple exit areas; and a plurality of anvils associated with the cartridge, the anvils having a first position toward the cartridge and a second position away from the cartridge. The stapler includes a plurality of bolsters, one of each of the plurality of bolsters associated with one of the plurality of anvils. The stapler includes a graft, wherein the graft is disposed between the anvils and the pushers, wherein the actuating mechanism may be utilized to advance the staples through the staple exit areas when the anvils are in the first position, such that the anvils interfere with the staples to assist the staples into loops capturing the graft, the bolster, and a vessel.

In one or more of the embodiments of the above stapler, the staples may be formed from a U-shaped section with elongate legs and a central portion connecting the legs. The staples may have a first condition in which the elongate legs are straight while stored in the stapler and a second condition, their natural condition in which the elongate legs are at least partially closed when discharged from an associated staple exit area. The staples and pushers may be arranged radially in a circle about a circumference of the cartridge. The pushers may advance along a longitudinal axis of the stapler to advance the staples and along the cartridge. The casing can move along a longitudinal axis of the stapler relative to the cartridge to cause the anvils to move between the first position and second position. The casing can advance toward the distal end of the stapler to cause the anvils to move to the first position. The casing can retract away from the distal end of the stapler to cause the anvils to move to the second position. At least a portion of at least one of the bolsters may be impregnated with a haemostatic agent. At least one of the anvils may be pivotally attached to the stapler to move between the first position and second position. At least one of the bolsters is attached to an inner surface of an associated anvil.

In another aspect of the second embodiment of the present application, disclosed is a method of attaching a graft to a vessel using a stapler of the second embodiment of the present application.

In an aspect of a third embodiment of the present application, disclosed is a surgical stapler that includes a casing with a plurality of staple exit areas; a cartridge within the casing, the cartridge has a split tip adapted to be spread between a closed position and an open position; a plurality of pushers mounted radially on the cartridge; a plurality of staples, one of the plurality of staples associated with each of the pushers; an actuating mechanism adapted to advance the pushers along the cartridge to push the staples through the staple exit areas; and an anvil associated with an exterior of the casing, the anvil has a first position away from said split tip and a second position toward said split tip. The stapler includes a bolster wherein the bolster associated with the anvil. The stapler includes a graft, wherein the graft is disposed between the casing and the cartridge, with a portion of the graft extending beyond and over the casing, wherein the actuating mechanism may be utilized to advance the staples through the staple exit areas when the split tip is in the open position and the anvil is in the second position such that the staples penetrate the graft, an inner wall of a vessel, an outer wall of the vessel, the bolster, and the graft for a second time, in that order.

In another aspect of the third embodiment of the present application, disclosed is a method of attaching a graft to a vessel using a stapler of the third embodiment of the present application.

### BRIEF DESCRIPTION OF THE DRAWINGS

The subject matter regarded as the invention is particularly pointed out and distinctly claimed in the concluding portion of the specification. The invention, however, both as to organization and method of operation, together with features, objects, and advantages thereof will be or become apparent to one with skill in the art upon reference to the following detailed description when read with the accompanying drawings. It is intended that any additional organizations, methods of operation, features, objects or advantages ascertained by one skilled in the art be included within this description, be within the scope of the present invention, and be protected by the accompanying claims.

In regard to the drawings,

FIG. 1 is a plan view of a stapler in accordance with an embodiment of the present invention;

FIG. 2 is a sectional view of the stapler of FIG. 1 showing the internal components thereof;

FIG. 3 is a longitudinal sectional view of the distal end of the staple housing forming a portion of the stapler of FIG. 1 showing the internal components thereof in accordance with a first embodiment of the present invention;

FIG. 4 is a perspective view of the distal end of staple housing of FIG. 3 showing the staple housing partially transparent for clarity;

FIG. 5 is a top view of the staple detent of the staple housing of FIG. 4;

FIG. 6A shows the staple of FIG. 3 in a natural condition;

FIG. 6B shows the staple of FIG. 3 in a deformed condition;

FIG. 7 is a longitudinal sectional view of the distal end of the staple housing of the stapler of FIG. 3 in an initial step in a vessel of a patient as part of a method of securing a graft to a vessel in accordance with one embodiment of the present invention;

FIG. 8 depicts a longitudinal sectional view of the distal end of the staple housing of the stapler in a further step of FIG. 7;

FIG. 9 depicts a longitudinal sectional view of the distal end of the staple housing of the stapler in a further step of FIG. 8;

FIG. 10 depicts a sectional view of the vessel attached to the graft following the withdrawal of the distal end of the staple housing of the stapler of FIG. 9;

FIG. 11 is a plan view of a stapler in accordance with a second embodiment of the present invention;

FIG. 12 is a partial cut-away perspective view of the distal end of the staple housing of FIG. 11;

FIG. 13A shows a detailed view of a staple used in the stapler of FIG. 12 in its natural condition; and

FIG. 13B shows a detailed view of a staple used in the stapler of FIG. 12 in its deformed condition.

FIG. 14 depicts a longitudinal sectional view of the distal end of the staple housing of the stapler of FIG. 12 in an initial position in a vessel of a patient as part of a method of securing a graft to the vessel in accordance with a second embodiment of the present invention;

FIG. 15 depicts a longitudinal sectional view of the distal end of the staple housing of the stapler in a further step of FIG. 14;

FIG. 16 depicts a sectional view of the vessel attached to the graft after the withdrawal of the distal end of the staple housing of the stapler of FIG. 15;

FIG. 17 is a partial cut-away sagittal view of a distal end of a staple housing for securing a graft to a vessel in accordance with a third embodiment of the present invention;

FIG. 18 is a distal end sectional view of the stapler of FIG. 17.

FIG. 19 is a partial cut-away perspective view of the distal end of the stapler in FIG. 17 in an initial position in a vessel of a patient as part of a method of securing a graft to a vessel in accordance with a third embodiment of the present invention;

FIG. 20 depicts a sectional view of the distal end of the staple housing of the stapler in a further step of FIG. 19;

FIG. 21 depicts a sectional view of the distal end of the staple housing of the stapler in a further step of FIG. 20;

FIG. 22 is a sagittal sectional view of FIG. 21;

FIG. 23 depicts a sectional view of the distal end of the staple housing of the stapler in a still further step of FIG. 22; and

FIG. 24 depicts a sectional view of the vessel attached to the graft following the withdrawal of the distal end of the staple housing of the stapler of FIG. 23.

### DETAILED DESCRIPTION

In the following is described the preferred embodiments of the staplers of the present invention. In describing the embodiments illustrated in the drawings, specific terminology will be used for the sake of clarity. However, the invention is not intended to be limited to the specific terms so selected, and it is to be understood that each specific term includes all technical equivalents that operate in a similar manner to accomplish a similar purpose.

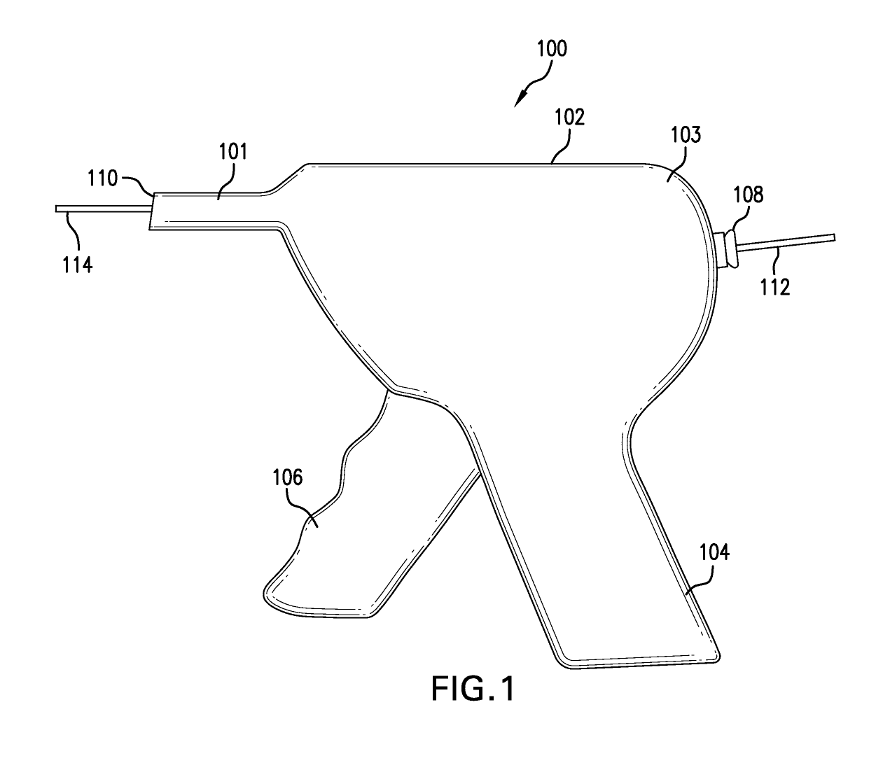

Referring to the figures, FIG. 1 depicts a stapler 100 in accordance with a first embodiment of the present invention. As explained in detail below, the stapler 100 is capable of performing an anastomoses between relatively small vessels and a graft such as a prosthetic vascular graft. As is shown, the stapler 100 may generally be shaped like a gun. The stapler 100 may comprise a housing 102 having a handle 104 and a trigger 106 extending therefrom. The housing may also include a barrel 101 having an output aperture 110. An input boss 108 may be located at the rear 103 of the housing 102. A guide wire 112 may extend into the input boss 108. Extending from the output aperture 110 may be a staple housing continuing to the distal end 114 of the stapler (not shown in FIG. 1).

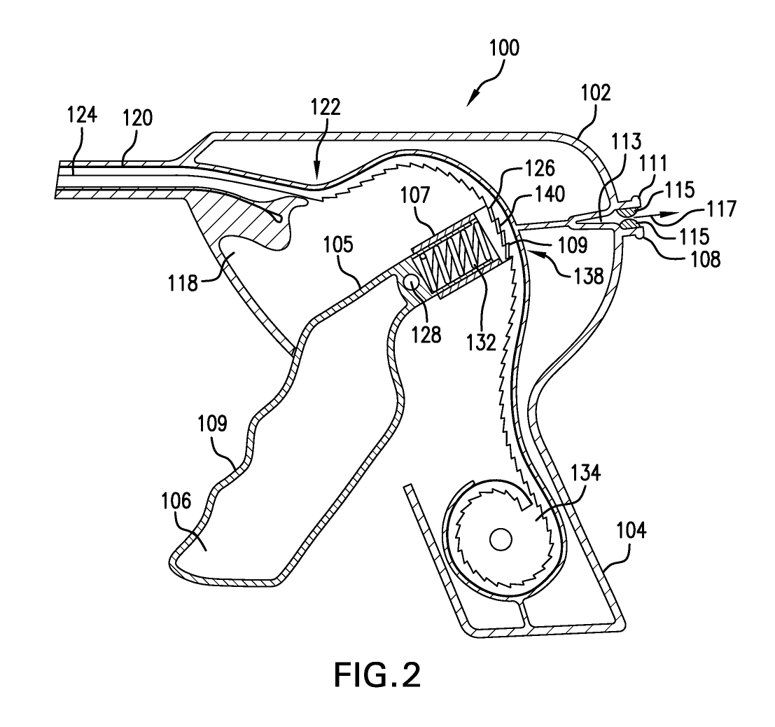

FIG. 2 depicts a cut-away view of the stapler 100 of FIG. 1. As shown, the trigger 106 may comprise an inner section 105 and an outer section 107. The inner section 105 may also include a grip 109, exterior to the housing 102. The inner section 105 may include a pin 128 attaching the trigger 106 to the housing 102, and about which the trigger may rotate. The trigger 106 may also include a spring mechanism (not shown) to bias the trigger 106 away from the handle 104. The outer section 107 of the trigger 106 may be attached to the inner section by a spring 132. Advantageously, the outer section 107 is permitted to shift relative to the inner section 105, to compress the spring 132. A toothed element 126 of the outer section 107 includes teeth 109 having sloped sections 138 and edges, or lips 140. Each of the sloped sections 138 of the teeth 109 assist with ratcheting action of the trigger 106, as will be discussed hereinafter.

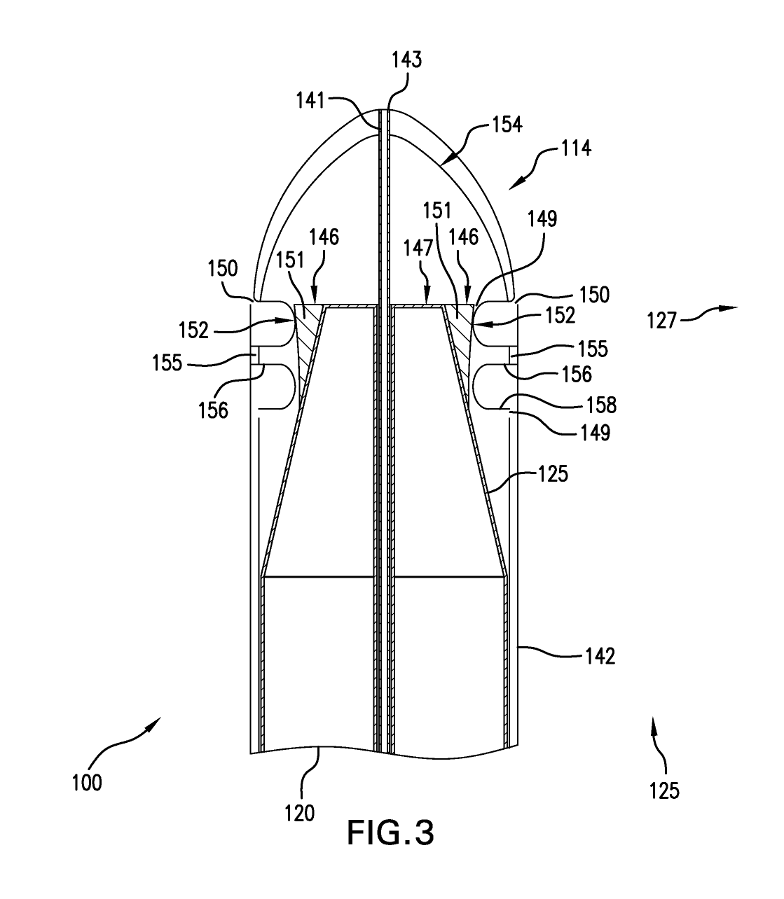

A ratcheted stapler actuator 120 may curve between the trigger 106 and a path created by the internal cavity 118 formed from the housing 102. The actuator 120 may include a ratcheted portion 122 at its trailing portion and a cylindrical portion 124 at its leading portion. The ratcheted portion 122 includes sloped sections 138 which may engage the toothed elements 126 of the stapler trigger 106. Upon actuation of the stapler trigger 106, which initiates rotation of the toothed elements 126 about pin 128, the actuator 120 may be displaced through the barrel 101 toward the distal end 114 (FIG. 3) of the stapler 100. As the trigger 106 is returned to its initial position, spring 132 permits ratcheting of the toothed elements 126 such that the actuator 120 remains in this advanced position. Portions of the ratcheted portion 122 of the actuator 120 may be stored in a spiral configuration within staging area 134, located within the handle 104 of the stapler 100.

Also shown in FIG. 2 are the internal components of the input boss 108. The input boss 108 comprises a flange 111 formed from the housing 102. The flange includes a cavity 113 extending into the internal cavity 118 of the housing 102. Within the cavity 113 near the flange 111 may be a pair of rubberized elements 115 having a boundary 117 therebetween. The guide wire 112 (FIG. 1) may be permitted to pass along this boundary from the exterior of the housing 102 to the internal cavity 118. Once inside the internal cavity 118, the guide wire may be permitted to extend through the barrel 101 into the guide wire channel 143 (FIG. 3) of the staple housing at the distal end 114, as will be discussed. In addition, the actuator 120 may be adapted to discharge staples 152 from the distal end 114 of the stapler (FIG. 3), as will be discussed below.

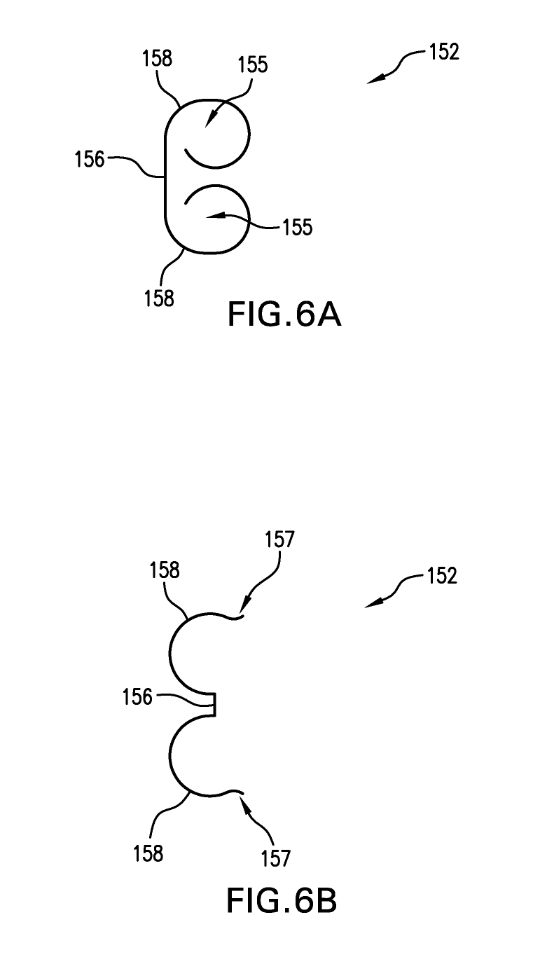

FIG. 3 is a longitudinal sectional view of the distal end 114 of the stapler 100 of FIG. 1, in accordance with a first embodiment of the present invention. The distal end 114 may include a stapler housing 142 within which the actuator 120 and pushers 146 may be configured. The pushers 146 include flanges 149 at opposite ends of the flat, a front surface 151 of the pushers which serve to hold the staples 152 in place during discharge. The actuator 120 and pushers 146 may include respective sloped surfaces 125, 147 such that advancement of the actuator 120 toward the distal end 114 of the stapler 100 (shown by arrow 123) forces the pushers 146 outward toward (shown by arrow 127) staple exit areas 150 formed through the stapler housing 142. The pushers 146 may be fixed in the longitudinal direction but capable of lateral movement in the direction shown by arrow 127. Outward movement of the pushers 146 forces staples 152 against staple detents 154 such that a central element 156 of the staple 152 may abut a holder portion 155 of the staple detent 154. This action permits opposed loops 158 of the staple 152 to close into a B-shaped form as the staple is deployed from the staple exit area 150 (See FIGS. 6A and 6B and corresponding description for further details). As a result of such action, the stapler 100 provides a staple 152 with a generally W-shape while stored in the stapler and a B-shape when discharged from the stapler.

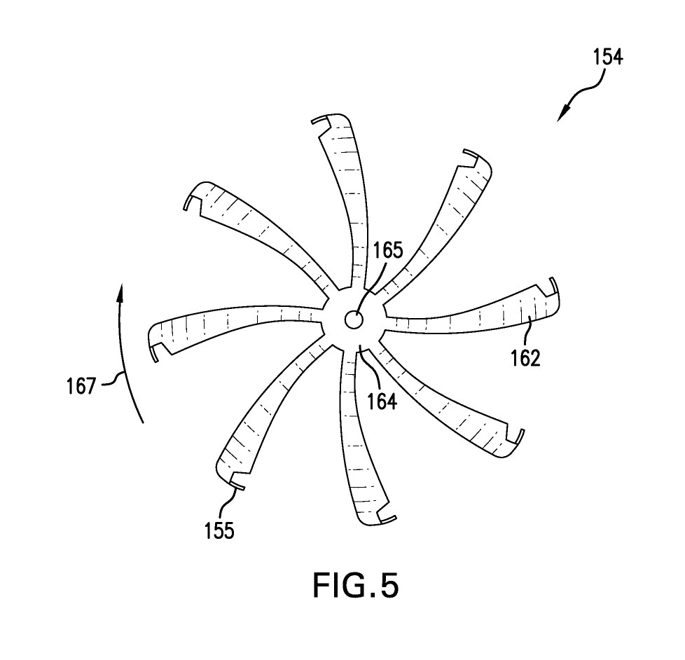

As the actuator 120 advances further toward the distal end 114, the pushers 146 displace the opposed loops 158 of the staples 152 laterally causing them to penetrate through and close around a vascular graft and vessel (not shown).The staple detent 154 may then be rotated a sufficient amount, for example approximately 15 degrees, to release the staples 152 from the associated staple exit area 150. For example, in one embodiment, the actuator 120 can include protuberances (not shown) on the sloped surface of the actuator adapted to engage the detent 154. In particular, the detent 154 can include arms 162 (see FIG. 5) such that each arm is associated with a protuberance. In operation, as forward displacement of the actuator 120 continues to move forward, the protuberances engage the arms 162 of the detent 154 and cause the detent to be simultaneously rotated laterally thereby releasing the staples from the staple exit areas 150. The stapler 100 and the internal components of the staple housing are preferably made of a biocompatible material well known in the art, such as various metals, plastics, composites, and the like.

Typically, the distal end 114 of the stapler 100 can be sized such that one staple 152 is fired per millimeter of vessel diameter. Accordingly, an 8 mm vessel will require use of a stapler 100 housing 8 staples 152 with an 8-armed staple detent 154. Of course, other sizes and combinations may also be utilized. For example, a 6 mm vessel will require use of a stapler 100 housing 6 staples 152 with a 6-armed staple detent 154, a 4 mm vessel will require use of a stapler 100 housing 4 staples 152 with a 4-armed staple detent 154, and a 10 mm vessel will require use of a stapler 100 housing 10 staples 152 with a 10-armed staple detent 154, as required for EU practice.

As will be discussed below, one full movement of the staple firing trigger 106 (FIG. 1) can cause the actuator 120 and pushers 146 to engage the staples 152 and discharge the staples through corresponding staple exit areas 150. Other firing mechanisms which do not resemble a trigger, but which provide a similar operation to control the actuator 120 are also contemplated. One example is a rotational dial which may be rotated a given number of turns to import the requisite translation of the actuators. Moreover, such actuators can be used to rotate the staple detent 154 the requisite amount to release the staples 152 once they are discharged.

The stapler 100 includes a guide wire channel 141 which extends along the entire length of the staple housing 114. The guide wire channel 141 provides a housing for guide wire 112 (FIG. 1), which is used to advance the distal end 114 of the stapler 100 to the location where the stapling is to be conducted.

Generally, advancement of the stapler 100 can be implemented as an “over the wire” type system. As an “over the wire” device, the distal end 114 of the staple housing 142 portion of the stapler 100 is designed to be guided through vessels following the path of a previously installed guide wire 112 (FIG. 1). For example, a guide wire 112 may be placed in an artery in a surgical procedure. The distal end 114 of the staple housing 142 may then be pushed along the length of the guide wire 112, which travels from a guide wire exit point 143 at the distal end 114, through guide wire channel 141 and out the input boss 108 of the housing 102 (FIG. 1). Once the distal end 114 reaches its destination, advancement may cease and the stapler 100 is ready to deploy staples 152.

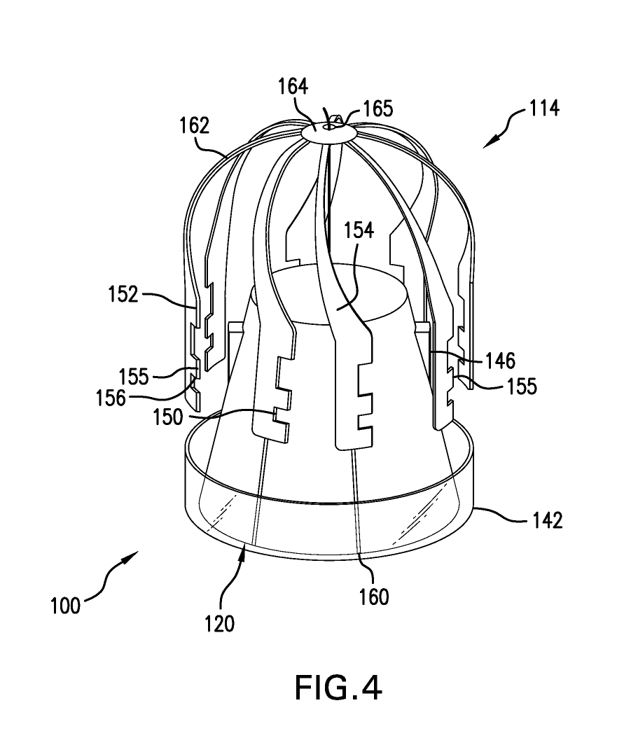

FIG. 4 depicts a perspective view of the stapler 100 with the stapler housing 142 of FIG. 3 being transparent for clarity. In this view, the radially aligned staples 152 are in view. It can also be seen how the cone shaped actuator 120 interacts with the pushers 146 to drive the pushers outward toward the staple exit areas 150. In this regard, the pushers 146 may ride in channels 160 formed in the actuator 120. The detent 154 is adapted to rotate between a first position (as shown in FIG. 4) and a second position. In the first position, the holder portion 155 of the detent 154 contacts the central element 156 of the staples thereby retaining the staples within the housing 142. In the second position, detent 154 rotates such that the holder portion 155 of the detent 154 no longer contacts the central element 156 of the staples thereby allowing the staples to be released through the staple exit areas 150. The detent 154 includes a centrally located opening 165 for rotatable attachment which may be disposed along a longitudinal axis of the stapler housing 142. As explained above, protuberances on sides of the actuator 120 can be used to rotate the detent. However, it is contemplated that the actuator can be manipulated to move the detent between the first and second position using an actuating mechanism such as a trigger or a dial or other well known actuating means.

FIG. 5 is a top view of the staple detent 154 of FIG. 4. The staple detent 154 may be formed from a series of arms 162 radiating from a central hub 164. As explained above, the detent 154 can be rotated between the first position to hold staples within the stapler and the second position to release the staples from the stapler. As such, the detent can be rotated (shown by arrow 167) about a longitudinal axis extending through the opening 165 using well known actuating means. For example, as explained above, the actuating means can include protuberances disposed on sides (sloped surface) of the actuator 120 adapted to engage the detent to cause the detent to rotate simultaneously laterally thereby releasing the staples, as explained above. Shown is an 8-armed staple detent 154 to support 8 staples for deployment in an 8 mm vessel. This arrangement allows one staple to be fired per millimeter of vessel diameter. Of course, other sizes and combinations may also be utilized.

FIGS. 6A and 6B depict the staple 152 used in the stapler 100 of FIG. 3. The staple 152 can be made of titanium, stainless steel, a memory alloy such as Nitinol, or other suitable material as is commonly used in the art. FIG. 6B shows the staple 152 having a generally W-shape while stored in the stapler. FIG. 6A shows the staple 152 having a generally B-shape when discharged from the stapler.

In the case of a staple made from a material other than memory alloy, within the staple housing 142 of the stapler (FIG. 3), the staple 152 will typically be in its natural condition shown in FIG. 6B, whereas FIG. 6A shows the staple 152 in its deformed condition, forming loops 159. As explained above, the staple is displaced laterally out through the staple exit sites while the detents hold unto the central portion 156 of the staple. This causes the staple to close into a B-shaped form (deformed condition as shown in FIG. 6A) as it exits the staple exit area. Thus, upon exiting the stapler, the staple 152 is deformed through the bending and shaping function applied by the stapler. In this case, the interaction of the actuator 120 and pushers 146 (FIG. 3) serves to control the formation of the staple to its deformed condition such that the desired layers of graft and vessel, as the case may be, are penetrated and possibly repenetrated to achieve the desired fixation

In the case of a staple made from a memory alloy, the staple 152 will typically be deformed into the condition shown in FIG. 6B, whereas FIG. 6A shows the staple 152 in its natural condition, forming loops 159 within the stapler. Upon exiting the stapler, the staple 152 may return to its natural condition owing particularly to the function of the memory metal, and not through bending or other shaping induced by the stapler. In the case of a memory metal, the interaction of the actuator 120 and pushers 146 (FIG. 3) serves to control the return of the staple to its default or natural condition such that the desired layers of graft and vessel, as the case may be, are penetrated and possibly repenetrated to achieve the desired fixation.

FIG. 6B shows the staple 152 comprising two U-shaped sections 158 connected together by central element 156. FIG. 6A shows the staple 152 in which the two U-shaped sections 158 form closed or partially closed loops 159, as the case may be. The staple 152 has spike ends 157 for piercing a vessel and a graft.

Regardless of the material of the staple 152, upon application into the graft and vessel utilizing the techniques to be discussed, the staple 152 forms the shape shown in FIG. 6A. As shown, the U-shaped sections 158 may be bent into loops such that the spiked ends 157 are adjacent to the central element 156. During the application process, the spiked ends 157 may pierce the graft and vessel so as to securely attach the two together. In other embodiments, the staple 152 may form closed loops 159 such that the spike ends 157 pierce the graft and vessel more than once.

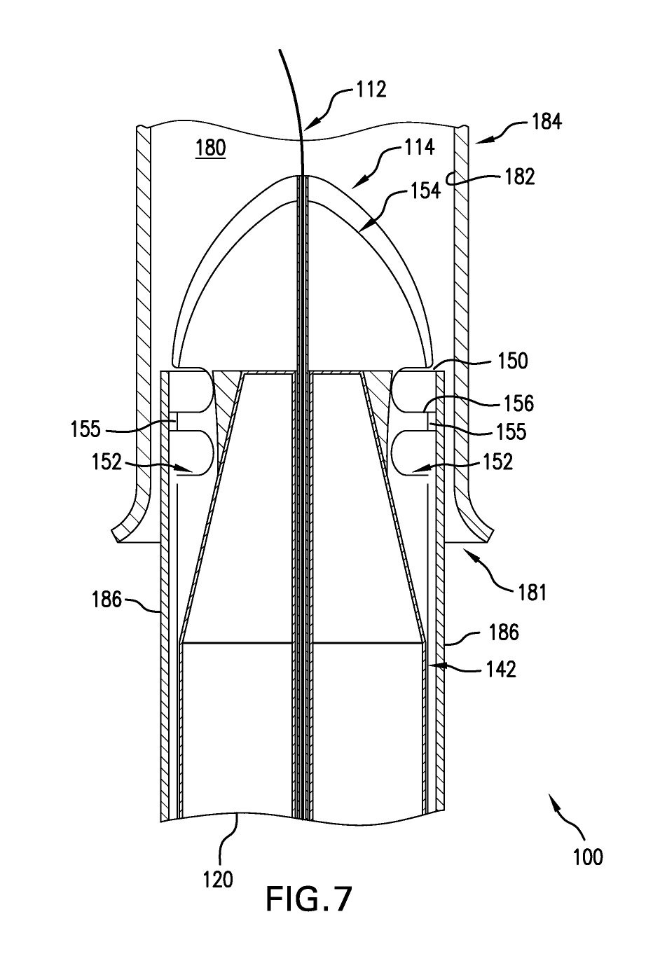

FIG. 7 is a longitudinal sectional view of the distal end 114 of the staple housing 124 of the stapler 100 of FIG. 3 in an initial step of a method or process of securing a graft such as graft 186 to a vessel 184 in accordance with one embodiment of the present invention. The graft 186 can be positioned adjacent an open end 181 of the vessel 184 using various means known in the art, such as through the use of a guide wire and visualization or location means. The staple exit areas 150 of the staple housing 142 are preferably positioned facing an inner wall 182 of the vessel 184. For example, the graft 186 can be mounted to the outer wall of the staple housing 142 using well known techniques such as hooks, barbs, adhesives or other attachment or delivery means. As explained above, the distal end 114 can be advanced into the interior of the vessel 180 using guide wire 112 and “over the wire” techniques well known in the art. In addition, the staple detent 154 is moved into the first position in which the holder portions 155 abut the central elements 156 of the staples 152 thereby holding the staples in place and preventing the staples from exiting the staple housing. As shown, the staple 152 has a generally W-shape while stored in the stapler and, as explained below, the shape of the staple changes to a generally B-shape when discharged from the stapler.

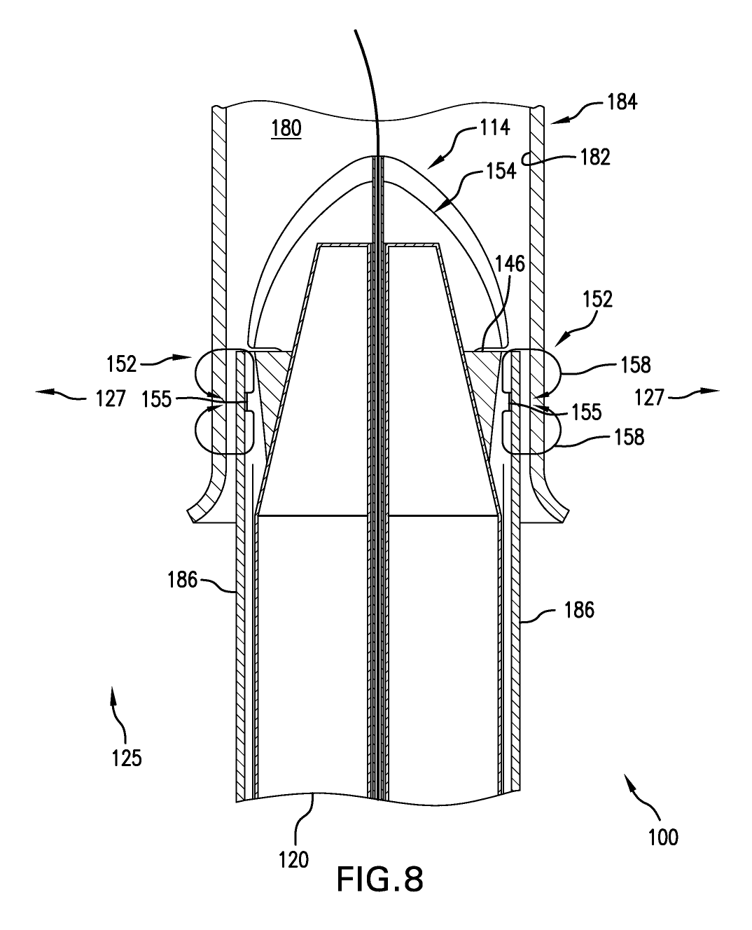

FIG. 8 depicts a longitudinal sectional view of the distal end 114 of the staple housing 142 of the stapler 100 in a further step of the process of FIG. 7. The actuator 120 is advanced forward toward the distal end 114 of the stapler 100 in the direction indicated by arrow 125. The actuator 120 can be advanced by actuating the trigger 106 of the stapler housing 104 (FIG. 1) with sufficient force to discharge the staples through corresponding staple exit areas 150. Such advancement of the actuator 120 forces the pushers 146 outward (in the directions indicated by arrows 127) toward the staple exit areas 150. Such outward movement of the pushers 146 forces the staples 152 against the staple detents 154 such that the central element 156 of the staple 152 abuts the holder portion 155 of the staple detent 154. This action permits the opposed loops 158 of the staple 152 to have a B-shape form as the staple is deployed from the staple exit areas 150. In other words, the staple changes from a W-shape while stored in the stapler to a B-shape as it is deployed from the stapler. As the actuator 120 is advanced further, the pushers 146 displace the opposed loops 158 of the staples 152 laterally causing them to penetrate through and close around the graft 186 and vessel 184.

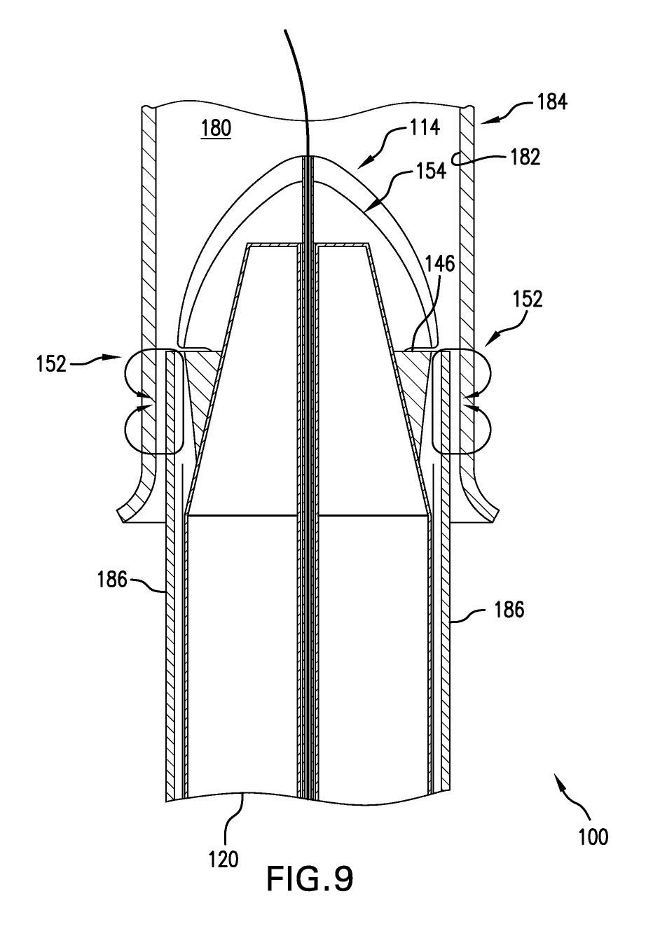

FIG. 9 depicts a longitudinal sectional view of the distal end 114 of the staple housing 124 of the stapler in a further step of the process of FIG. 8. Once the staples 152 have penetrated the vessel 184 and the graft 186, the staples 152 are ready to be released from the stapler. As such, the staple detent 154 is then placed in the second position by rotating in an amount sufficient, for example approximately 15 degrees, to move the holder portion 155 away from the central element 156 of the staples thereby releasing the staples 152 from the staple exit areas 150. In one embodiment, the trigger 106 of the stapler 100 (FIG. 1) advances the actuator 120 as well as rotates the detent which causes the release of the staples. As explained above, the stapler can include protuberances on sides of the actuator 120 adapted to engage the detent to cause the detent to rotate simultaneously laterally thereby releasing the staples. At this stage of the process, the staples 152 have been discharged to secure the graft 186 and the vessel 184 to each other and the distal end 114 of the stapler 100 can now be withdrawn from the vessel and graft.

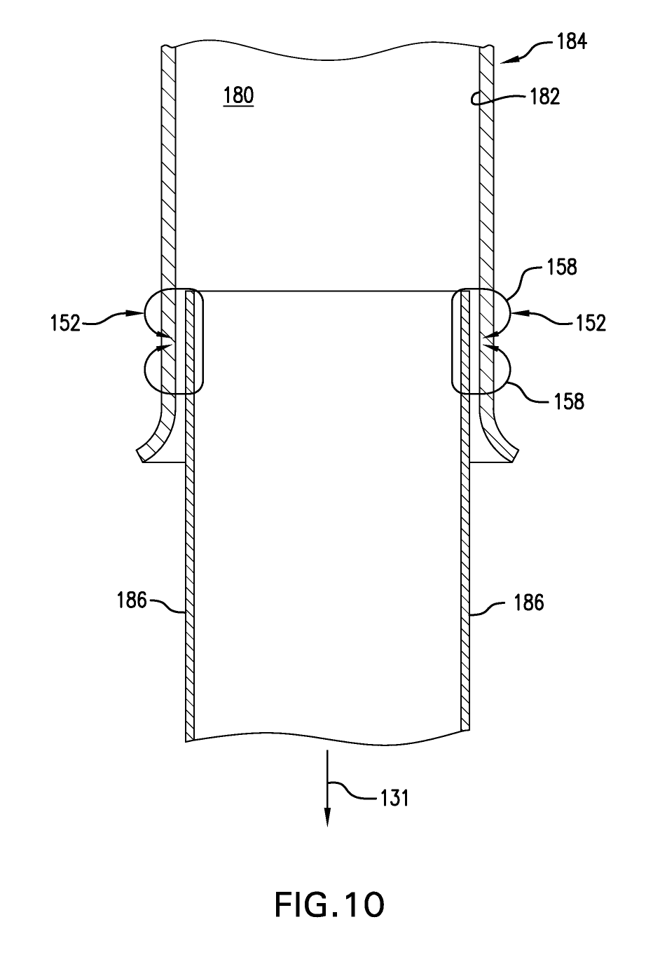

FIG. 10 depicts a cross-sectional view of the vessel attached to the graft after the withdrawal of the distal end of the staple housing of the stapler of FIG. 9 in the direction shown by arrow 131. As shown, the techniques of the present invention provide a tight relation between the graft 186 and vessel 184 using staples 152. In particular, each of the U-shaped sections 158 of the staples 152 penetrates the graft 186 and vessel 184 to provide a secure connection to each other. Although shown as penetrating the vessel and graft only once respectively, it will be appreciated that in other embodiments, the staples may fully close and pierce each more than once. For example, the staples 152 can penetrate the graft for a second time, as the case may be.

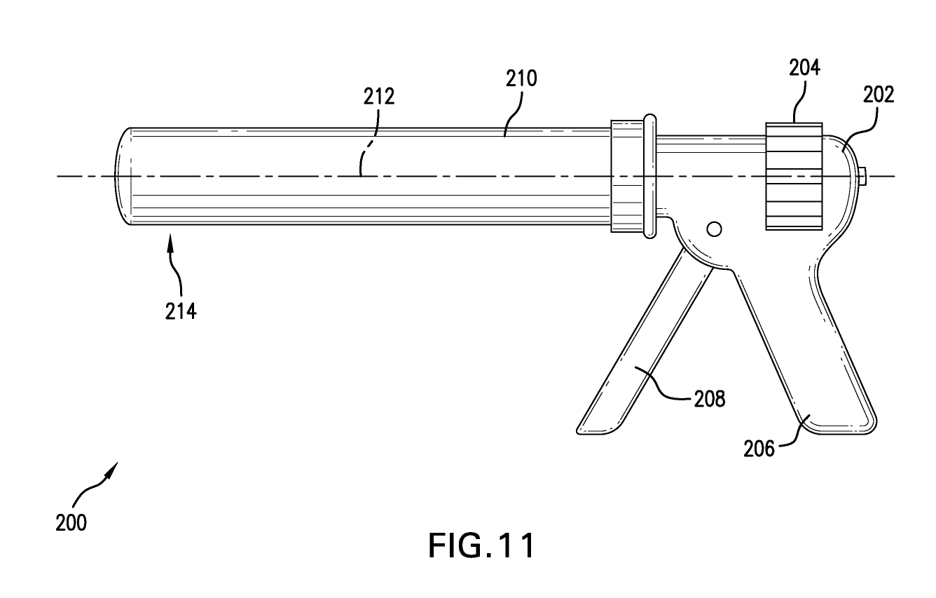

FIG. 11. is a plan view of a stapler in accordance with a second embodiment of the present invention. FIG. 11 depicts a side view of a stapler 200 adapted for use in end-to-side or end-to-end vascular anastomosis. The stapler 200 comprises a housing 210 with a proximal end 202 and a distal end 214. The proximal end 202 includes a handle 206 for manipulation of the instrument during a surgical procedure. The proximal end 202 also includes a staple firing trigger 208 to facilitate engagement of the staples (not shown) in the end-to-side anastomosis. The housing 210 extends from the proximal end 202 along a longitudinal centerline 212 toward its distal end 214. An actuating dial 204 may protrude from the housing 210 at the proximal end 202 of the stapler 200. As explained below, the dial 204 and the trigger 208 can serve as actuating mechanisms to operate the stapler including discharging staples therefrom.

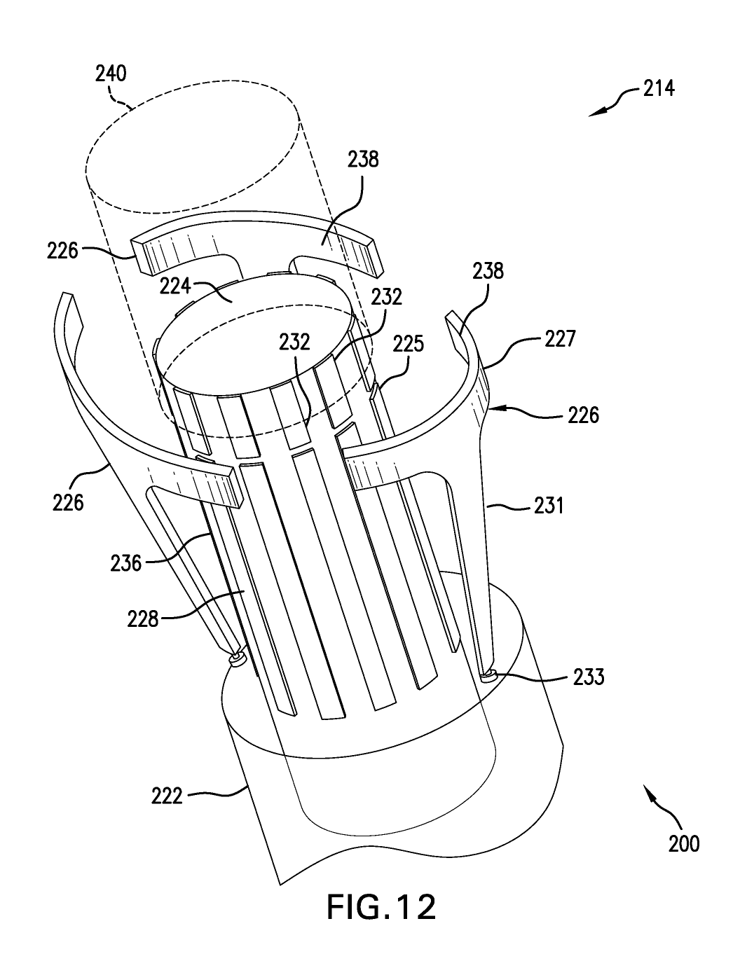

FIG. 12 is a partial cut-away perspective view of the distal end 214 of the stapler, configured to provide end-to-end anastomosis. The stapler 200 may comprise an outer housing 222 (portions of which are cut away for clarity) with a cartridge 224 therein. Between the cartridge 224 and the outer housing 222, the stapler 200 may include an anvil 226 disposed about the circumference of the cartridge. In one embodiment, the anvil 226 can be the tripartide anvil shown in FIG. 12. Anvils with more or less than three parts may also be utilized. The anvils 226 have a generally T-shape formed from a top horizontal member 227 and a bottom vertical member 231 coupled to the top member at about the midway point of the top member. The top member 227 is generally curved inward toward the cartridge 224 complementing the curved shape of the cartridge 224. The top member 227 has an inner surface facing the cartridge 224 and adapted for holding bolsters 238. The bottom member 231 has a bottom portion 233 for pivotal attachment to the outer wall of the cartridge 224. The anvils 226 are adapted to move between a closed position toward the cartridge 224 and an open position away from the cartridge, during operation as explained below. It will be appreciated that FIG. 12 depicts the anvils 226 in at least a partially open position. As shown, the bolsters 310 are mounted onto the inside surface of the anvils 226 to form a ring shape facing or surrounding the cartridge 224. The anvils 226 lack pockets and can be used to close the staples without requiring registration between the staples and anvil pockets. The configuration of the anvils 226 provides circumferential counter pressure which holds the graft and vessel wall in place to be penetrated by the staples.

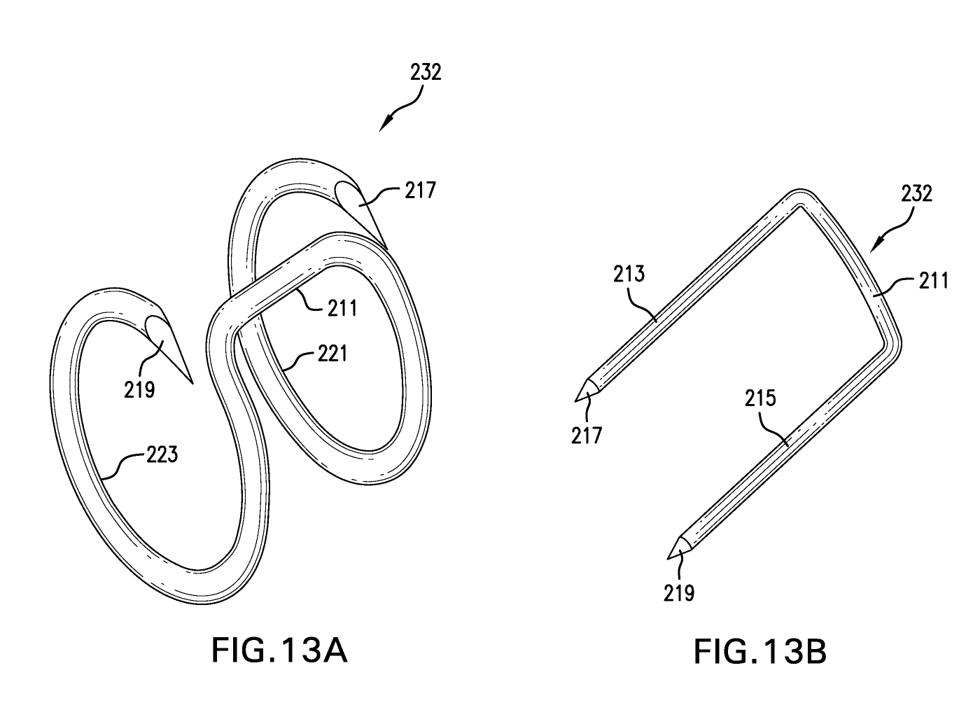

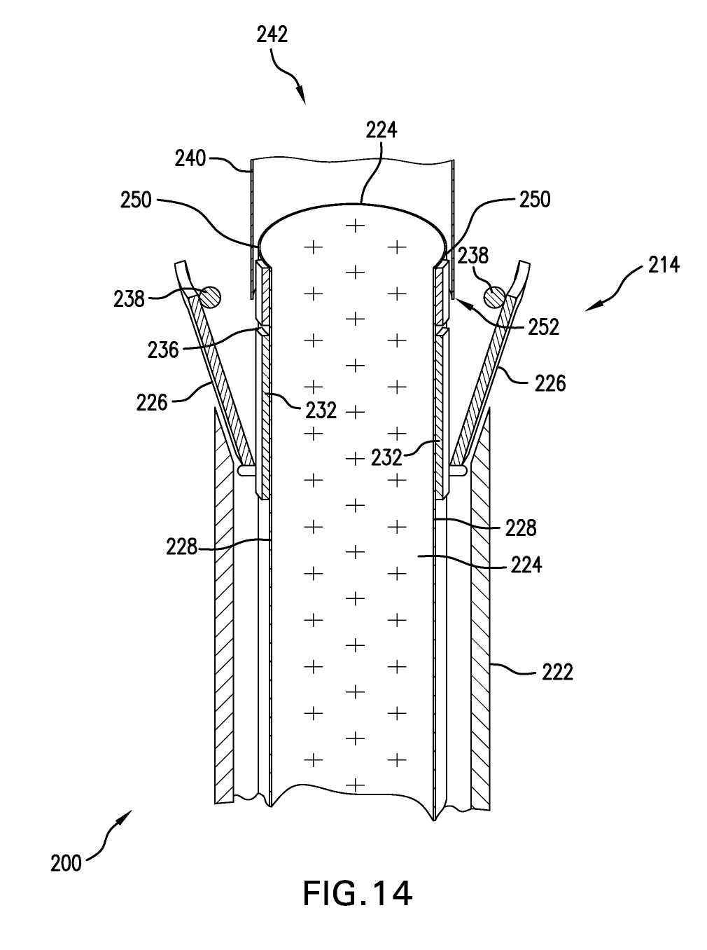

The cartridge 224 may be associated with a series of pushers 228 disposed radially about the circumference of the cartridge 224. The pushers 228 are adapted to move longitudinally along the stapler 200 relative to the cartridge 224. At the distal end 229 of each pusher, there may be configured a fastener, or staple 232. The stapler 200 includes staple exit areas 250 (best seen in FIG. 14) for allowing the staples 232 when discharged to exit from the stapler. The staples 232 are preferably formed from a memory metal and are installed in their deformed state, such that they form a U-shape with elongated legs and a central portion connecting the legs (See FIGS. 13A and 13B and corresponding description for further details). In a natural condition, the staples 232 may be formed to open loops, closed loops, or the like, as will be discussed. The staples 232, like the pushers 228, are arranged radially in a circle along the circumference of the cartridge 224 located along the stapler's inner wall.

A graft 236 is mounted between the pushers 228 and the anvils 226. The graft 236 is a generally elongated cylindrical structure with a generally circular cross-section for attachment to a vessel 240, such as a blood vessel, during a surgical procedure. The graft 236 is a specialized vascular prosthesis typically manufactured from Dacron®, PTFE, or other suitable material useful for such purposes. Dacron® is a registered trademark of E.I. DuPont de Nemours and Company, 1007 Market Street, Wilmington, Del. 19898.

The bolsters 238 are prosthetic components utilized to help support the graft 236 after installation of the graft, and particularly to fill voids between the graft, staple 232, and vessel 240, to ensure a water tight seal. The bolsters 238 may have an elongated O-ring shape and are mounted on an inner surface of the top member 227 of the anvil 226 facing the cartridge 224. The bolsters 238 are formed separately from the graft 236, and are typically made from Teflon® or Dacron®. Both Teflon® and Dacron® are registered trademarks of E.I. DuPont de Nemours and Company, 1007 Market Street, Wilmington, Del. 19898. The bolsters 238 may also be made from a fiber material, such as polymer fiber, or a modified bovine pericardium strip. The bolsters also can be made from any material which is suitable for surgery and which meets the objectives set forth herein.

The bolsters 238 can be attached to the anvil 226 using any biologically compatible means (physical connection, chemical adhesive, etc.), including heated fusion or chemical bonding. The bolsters 238 are typically a ring of material having a diameter greater than the thickness of the vessel to which it is to be attached. To facilitate healing following the surgical procedure, the bolsters 238 may be impregnated with a haemostatic agent. However, the risk of the haemostatic agent leaching into the vessel from the bolsters 238 may render such impregnation too risky. Haemostatic agents are known in the art. As will be shown, the bolsters 238 may be placed into position for anastomosis by movement of the anvil 226.

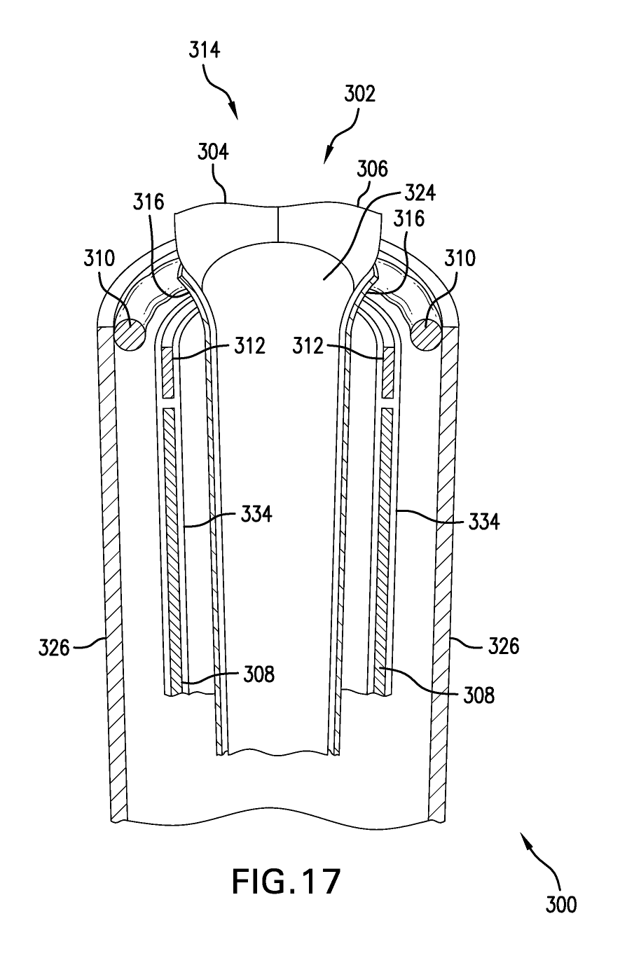

FIGS. 13A and 13B show a detailed view of the staple 232 used in the stapler 200 in FIG. 12. The description of the staple 232 also applies to the staple 312 used in the stapler 300 in FIG. 17 described below. As shown in FIG. 13B, the staple 232 may be predominantly U-shaped in its deformed condition and may comprise a pair of legs 213, 215 connected by a central portion 211. Each of the pair of legs 213, 215 may terminate with spiked ends 217, 219.

Upon application into the graft and vessel utilizing the techniques described herein, the staple 232 may be permitted to return back to its natural condition, for example into the shape shown in FIG. 13A. As shown, the legs 213, 215 may be bent into loops 221, 223 such that the spiked ends 217, 219 are adjacent to the central portion 211. During the application process, the spiked ends 217, 219 may pierce the graft and vessel so as to securely attach the two together. In other embodiments, the spiked ends 217, 219 may continue to loop past the position shown in FIG. 13a, such that the loops fully close and such that the vessel and graft may be repenetrated.

As explained above, preferably, the staple 232 may be constructed of a memory alloy such as Nitinol, as is commonly used in the art. Within the staple housing of the stapler, the staple 232 will typically be deformed into the condition shown in FIG. 13B. Whereas FIG. 13A shows the staple 232 in its natural condition. The staple 232 may be formed so as to create loops 221, 223, as shown in FIG. 13A, in its natural condition. If so formed, the staple 232 should therefore be straightened prior to insertion into the stapler. Upon exiting the stapler, the staple 232 may then return to its natural condition owing particularly to the function of the memory metal, and not through bending or other shaping induced by the stapler.

FIG. 14 depicts a sagittal cross-sectional view of the stapler 200 of FIG. 12 in a first step of securing the graft 236 to the vessel 240. As shown, the distal end 214 of stapler 200 may be brought into a cavity 242 of an open-ended vessel 240 (e.g., blood vessel) with the anvils 226 (open position) spread apart away from the cartridge 224 in the first position. Preferably, the stapler 200 is inserted into the cavity 242 of the vessel such that the staple exit areas 250, where the staples 232 are deployed upon firing, are just beyond the limit of the end 252 of the blood vessel 240 within the cavity 242. It will be appreciated that while in this position, the staples 232 are capable of penetrating the vessel 240 as they are deployed from the staple exit areas 250.

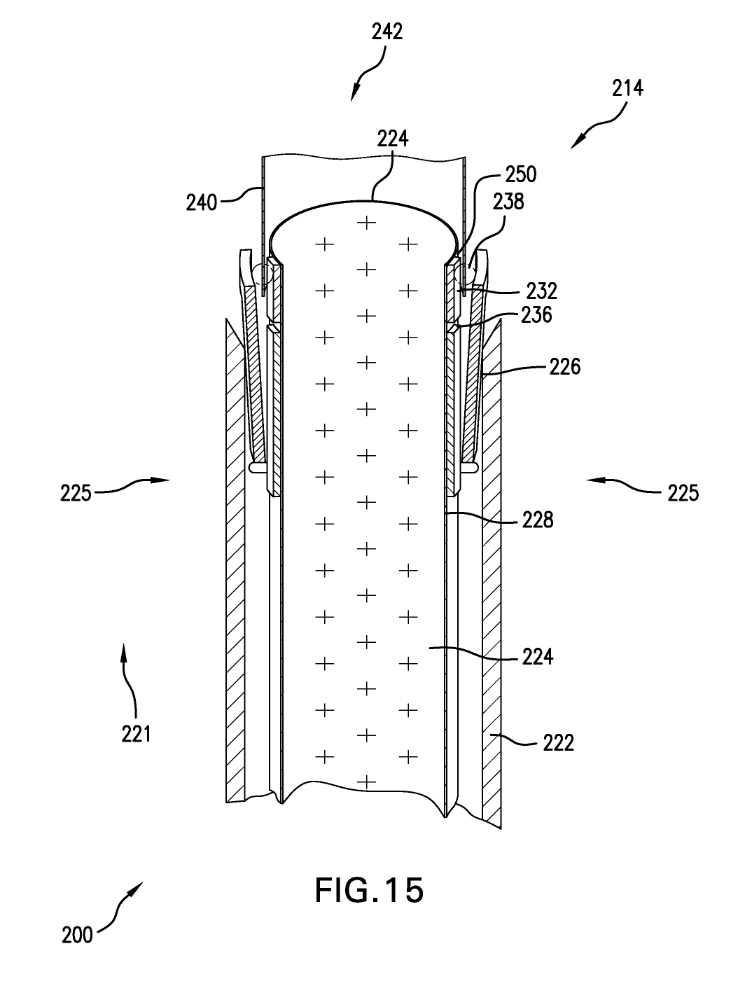

FIG. 15 depicts a sagittal cross-sectional view of the stapler 200 of FIG. 14 after deploying the staples 232. Deployment of the staples 232 may be achieved by first placing the anvils 226 in the closed position. In one embodiment, this can be achieved by advancing the outer casing 222 upward toward (direction indicated by arrow 221) the distal end 214 of stapler 200 which forces the anvils 226 inward toward (direction indicated by arrow 225) the cartridge 224. Advancement of the outer casing 222 may be achieved by actuating the trigger 208 or the dial 204 of the stapler 200 (FIG. 11) or other actuation mechanism well known in the art.

After closing of the anvil 226, the staples 232, each of which being associated with a pusher 228, may be deployed. To achieve deployment, the pushers 228 may be driven forward toward (direction indicated by arrow 221) the distal end 214 of the stapler 200 by an actuator such as the trigger 208 or the dial 204 of the stapler 200 (FIG. 11) or other actuation mechanism well known in the art. The actuator preferably displaces all pushers 228 forward simultaneously when an actuating mechanism, such as the trigger, is pulled. Other actuating mechanisms include advancement screws or other known advancement devices. It is preferred that the actuating mechanism quickly and efficiently advance the pushers 228 through the required excursion to deploy the array of staples 232 simultaneously.

The distance traveled by the pushers 228 is preferably sufficient to completely displace the staples 232 which then penetrate through the graft 236 and vessel 240, entrapping the bolsters 238 and the graft 236 on their way to preferably closing completely to a preformed double loop with a predetermined diameter. The staples 232 may also close an incomplete loop, as engineered for the particular application. It will be appreciated that the staples may be formed to have loops with various predetermined diameters particularly configured to associate properly with vessels of varying wall thickness and different sized bolsters.

As shown, the outer casing 222 is in the fully advanced position such that the outer casing 222 forces the anvils 226 inward toward the cartridge 224 (closed position). Further, the pushers 228 have been fully advanced to push the staples 232 through the staple exit areas 250. The anvils 226 have also interfered with the staples 232 upon exiting of the staples 232 from the staple exit areas 250, to assist with shaping the staples 232 into their preformed closed loops or partially open loops, as the case may be. As the staples 232 are driven from the staple exit areas 250, the staples penetrate the graft 236, bolsters 238, and vessel 240, to secure the graft 236 and bolsters 238 to the vessel 240. It will also be noted that the bolsters 238 abut the vessel 240 so as to achieve a tight relation between the now connected graft 236, bolsters 238, and vessel 240.

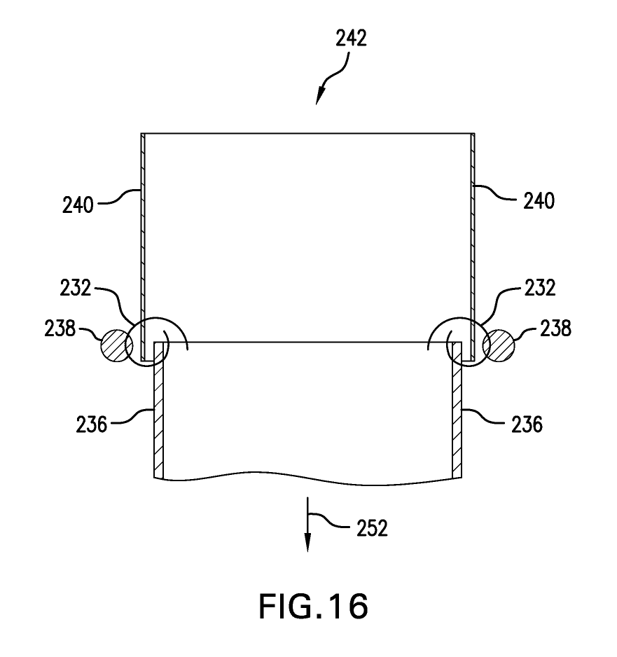

FIG. 16 depicts a cross-sectional view of the vessel attached to the graft after the withdrawal of the distal end of the staple housing of the stapler of FIG. 15. Once the stapler has deployed staples 232 to penetrate and connect graft 236, bolsters 238, and vessel 240 together, the surgical procedure is almost complete and the stapler can be removed or withdrawn from the patient. As shown, the techniques of the present invention provide a tight relation between the graft 236, bolsters 238, and vessel 240. The stapler can be removed by retracting the distal end of the stapler away from the interior cavity 242 of the vessel 240 in the direction shown by arrow 252.

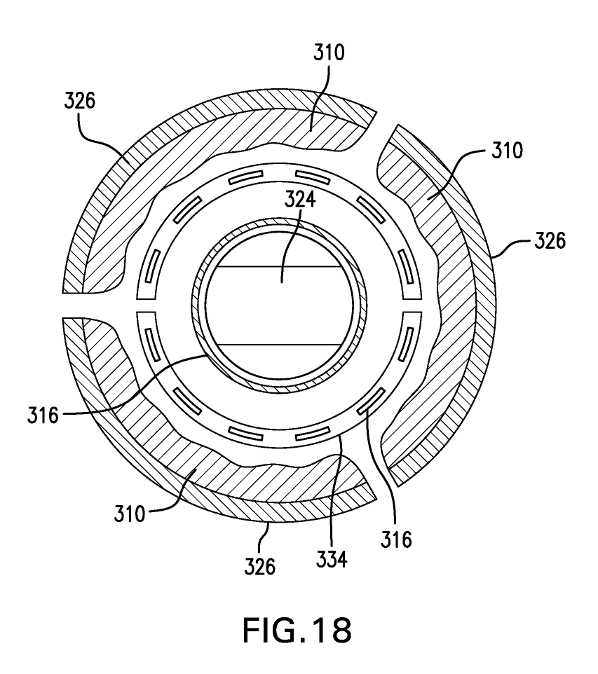

FIG. 17 is a partial cut-away sagittal view of a distal end 314 of a staple housing of a stapler 300 in accordance with a third embodiment of the present invention. FIG. 18 shows the distal end 314 of the stapler 300 in sectional view. The stapler 300 is configured .to provide end-to-side anastomosis. The distal end portion 314 of the stapler 300 is only shown. However, it should be understood that the stapler 300 can have a housing at a proximal end such as that shown in FIG. 11 or FIGS. 1 and 2, and described above, or other well known means.

Sizing of an arteriotomy to match the selected stapler size is important. The stapler 300 is configured such that staples 312 (fasteners) follow a centrally located cartridge 334 (typically within channels) to return to their preset shape and size. These techniques may also be used to construct staplers suitable for end-to-side anastomoses. This approach may be used to build end-to-side staplers by beveling the front end (at the distal end) of the stapler.

As shown in FIGS. 17 and 18, the stapler 300 may comprise many of the same components as stapler 200 of FIG. 12, including pushers 308 adjacent staples 312, anvils 326, and a cartridge 334 to support the staples. The stapler 300 may also include a split tip 302, having a first section 304 and a second section 306, and an inner casing (central shaft) 324, which is positioned between the pushers 308 and the cartridge 334. It is preferred that the cartridge 334 and body of the stapler 300 be beveled, such as, for example, 30 degrees.

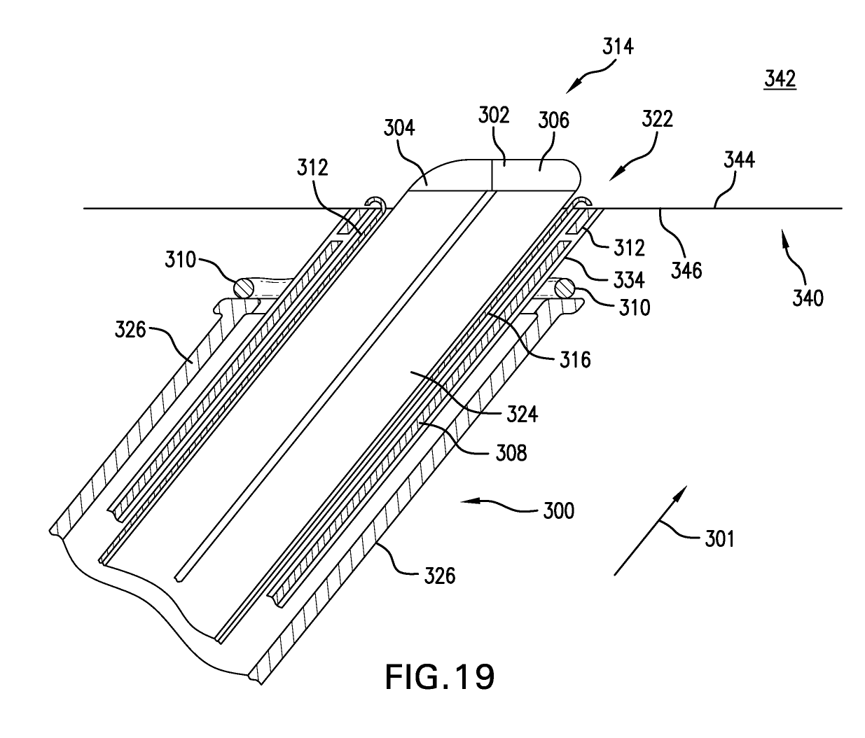

In the adaptation for use in end-to-side anastomoses, the stapler is configured for insertion into a cavity 342 of a vessel 340 through an arteriotomy. As will be discussed more fully with respect to FIGS. 19-24, the split tip 302 at the distal end 314 of the stapler may be placed in an open position with the split tip spread apart and the stapler 300 retracted away from the vessel 340 to associate the split tip with the inner wall 344 of the vessel. The anvils 326 can then be advanced toward the vessel to abut the bolsters 310 against an exterior wall 346 of the vessel, squeezing a curled end portion 322 of a graft 316 between the split tip 304 and the vessel wall. The staples 312 may then be deployed to engage the walls 344, 346 of the vessel, also penetrating through the graft 316 and bolsters 310. The split tip 304 may then be placed in a closed position in which the split tip is brought together and the stapler 300 removed, leaving the graft 316 and bolsters 310 behind, attached to each other and to the vessel.

The actuation of the various components of the stapler 300 can be achieved using any of the actuating mechanisms described above or techniques well known in the art. For example, the staples 312 can be advanced forward toward the distal end of the 314 of the stapler and thereby discharged by using an actuator, such as actuator 120 as used in stapler 200. The split tip 302 can be spread (separate sections 304, 306) and retracted using similar techniques. Likewise, the anvils 326 can be advanced and retracted using similar techniques.

Turning to a first step of a surgical procedure involving end-to-side anastomoses, shown in FIG. 19, the distal end 314 of the stapler 300 is advanced toward the vessel (shown by arrow 301) and inserted into the cavity 342 of the vessel 340, which has been formed by a longitudinal incision. The stapler is inserted such that the split tip 302 is in a closed (non-spread) condition. The stapler 300 is preferably inserted such that the curled end 322 of the graft 316 is within the cavity 342. It will be appreciated that the curled end 322 of the graft 316 is formed simply by extending the graft 316 over and beyond the limits of the cartridge 334.

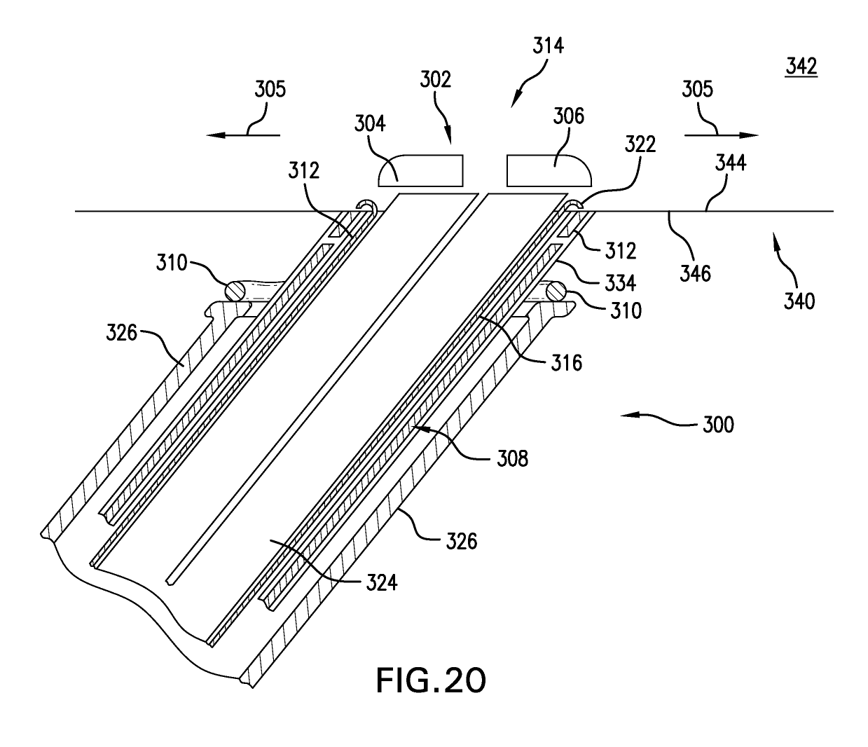

FIG. 20 shows a further step of the surgical procedure of FIG. 19 involving connecting graft 316 to vessel 340 in an end-to-side relation. The split tip 302 is then placed in the open position (split tip spread apart) such that the first section 304 and second section 306 separate outward, as indicated by arrows 305, while inside the cavity 342 of the vessel. In this regard, it is preferred that the longitudinal incision in the vessel 340 be of a length greater than that of the diameter of the casing 324, but less than that of the diameter of the spread apart split tip 302 such that the stapler 300 may be pulled back from within the cavity 342 to abut the split tip 302 against the inner wall 344 of the vessel 340 with the curled end 322 of the graft 316 captured therebetween.

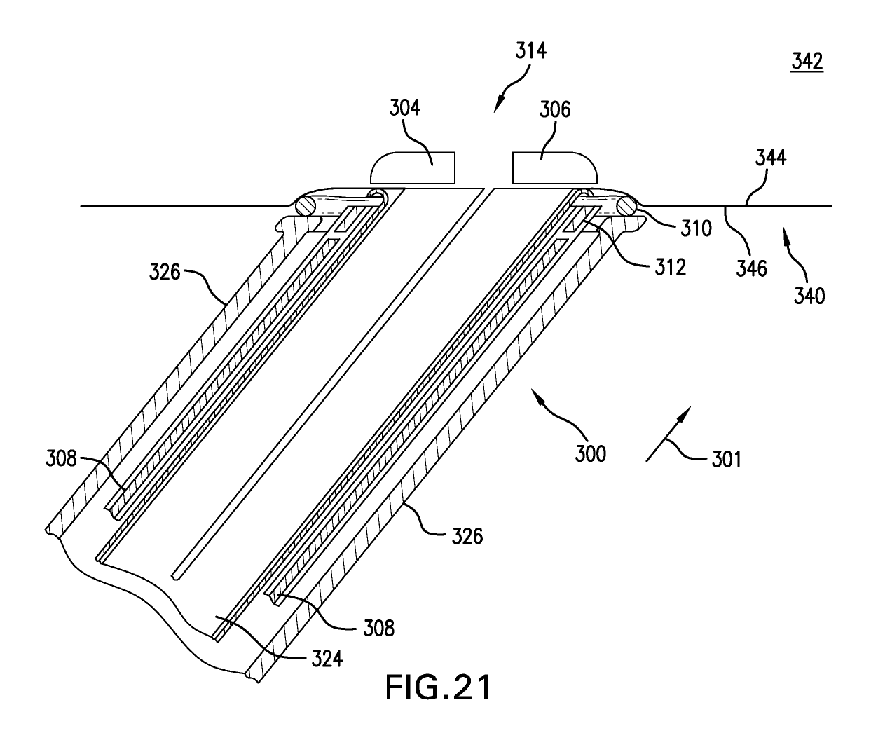

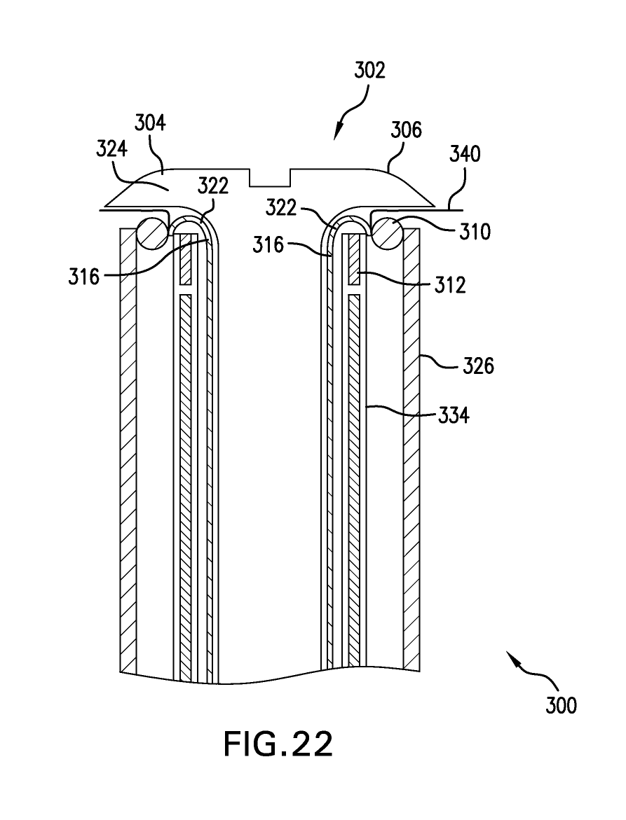

FIG. 21 shows a further step of the surgical procedure of FIG. 20 involving connecting the graft 316 to the vessel 340 in an end-to-side relation. FIG. 22 shows a sagittal view of the stapler used in the step of FIG. 21. Once the split tip 302 has been separated into first section 304 and second section 306, the anvils 326 may then be advanced toward the distal end 314 of the stapler as indicated by arrow 301. Preferably, the anvils 326 are slid along the length of the stapler 300 toward the split tip 302 to push the bolsters 310 against the outer wall 346 of the vessel 340. In this position, the staples 312 may be discharged (fired) such that they penetrate, in order, the curled end 322 of the graft 316, the inner wall 344 of the vessel 340, the outer wall 346 of the vessel 340, the bolster 310, the graft 316 for a second time, and then curl to the extent engineered for the application to repenetrate at least some of the aforementioned elements. The discharge of the staples 312 can involve advancing the pushers 308 forward toward (as indicated by arrow 301) the distal end 314 of the stapler sufficient to discharge the staples as required above. This can be achieved using actuating mechanisms as explained above and well known in the art. It will be appreciated that the staples 312 can be configured to penetrate the vessel 340, graft 316 and bolster 310 in other ways, as the case may be. For example, the staples 312 can contact and/or surround the bolster 310 without having to penetrate the bolster while still providing a tight relation.

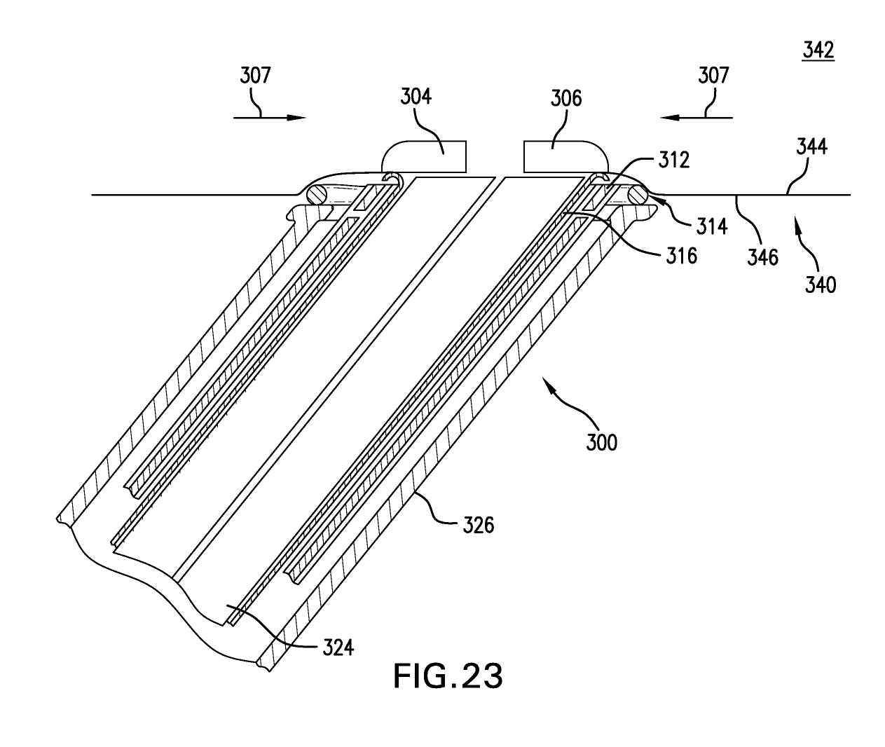

FIG. 23 shows a further step of the surgical procedure of FIG. 22 involving connecting the graft 316 to the vessel 340 in an end-to-side relation. The deployment of staples 312 in the above manner results in the graft 316 being attached to the vessel 340 in an end-to-side relation, as shown. The split tip can then be moved to the closed position in the direction shown by arrow 307. Preferably, the first section 304 and second section 306 of the split tip 302 are brought back together, and the stapler 300 can then be removed from within the vessel 340, leaving the now attached graft 316 behind.

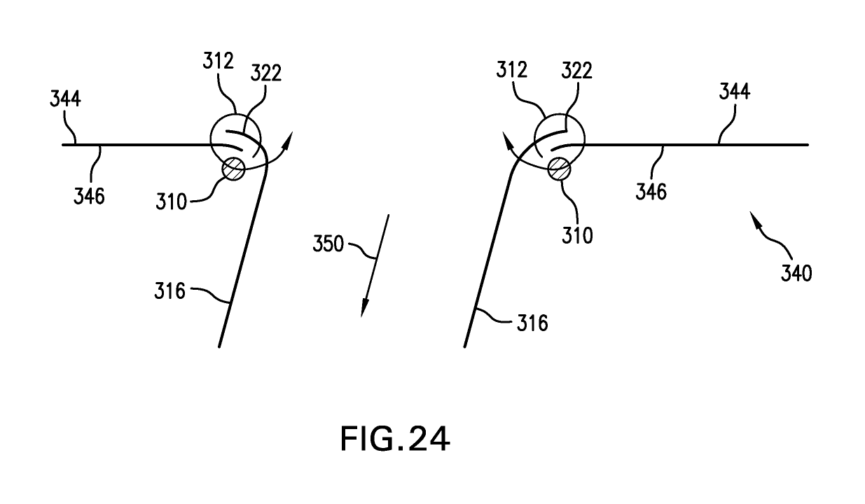

FIG. 24 shows a detailed view of the vessel attached to the graft after the withdrawal of the distal end of the staple housing of the stapler of FIG. 23. The stapler 300 is withdrawn by retracting the stapler in the direction indicated by arrow 350. As shown, the staples 312 have penetrated, in order, the curled end 322 of the graft 316, the inner wall 344 of the vessel 340, the outer wall 346 of the vessel 340, the bolster 310, the graft 316 for a second time, and then curl to the extent engineered for the application to repenetrate at least some of the aforementioned elements. Such techniques help provide a tight relation between the graft 316, vessel 340 and bolster 310.

Although the invention herein has been described with reference to particular embodiments, it is to be understood that these embodiments are merely illustrative of the principles and applications of the present invention. It is therefore to be understood that numerous modifications may be made to the illustrative embodiments and that other arrangements may be devised without departing from the spirit and scope of the present invention as defined by the appended claims.

1. A stapler for simultaneously discharging a series of staples, said stapler comprising:
an outer housing adapted to fit completely within an interior of a vascular graft;
an actuator and a plurality of pushers configured within said outer housing such that forward advancement along said outer housing of said actuator forces said pushers radially outward toward said outer housing;
a plurality of staples;
a staple detent having a plurality of arms, each of said arms securing one of said plurality of staples against one of said pushers; and
a plurality of staple exit areas formed through said outer housing, wherein one said staple exit areas is associated with a staple,
wherein said actuator may be advanced to force said pushers radially outward to discharge said staples through said staple exit areas.

2. The stapler of claim 1 wherein said stapler discharges staples adapted to penetrate a vessel wall and a graft wall to secure said graft to said vessel.

3. The stapler of claim 1 wherein said staples have a generally W-shape while stored in the stapler and a generally B-shape when discharged from the stapler.

4. The stapler of claim 1 wherein at least one of said staples is made from at least one of a memory alloy, titanium and stainless steel.

5. The stapler of claim 1 wherein at least one of said staples is formed from a plurality of opposed loops connected by a central element.

6. The stapler of claim 5 wherein said at least one staple has a first condition in which said opposed loops are open while stored in the stapler and a second condition in which said opposed loops are at least partially closed when discharged from an associated staple exit area.

7. The stapler of claim 1 wherein said staples are disposed in a circle about a circumference within said outer housing.

8. The stapler of claim 1 wherein said staple detent is adapted to rotate between a first position to retain said staples within said outer housing and a second position to release said staples from said staple exit areas.

9. The stapler of claim 1 further comprising a graft mounted on said outer housing such that the distal end of the stapler outer housing fits entirely within the interior of the graft and the vessel and positioned such that the staples, when discharged, penetrate said graft and the vessel wall.

10. A method of attaching a graft to a vessel using a stapler comprising an outer housing at a distal end of the stapler, a graft disposed upon the outer housing, a staple detent with a plurality of arms, each of the arms retaining one of a plurality of staples, the detent adapted to release the staples from an associated staple exit area, and a staple actuating mechanism to discharge the staples through an associated staple exit area, the method comprising:
inserting the distal end of the stapler within the vessel such that the staple exit areas and the graft are adjacent the vessel;
discharging the staples from the staple exit areas and to cause the staples to penetrate the graft and the vessel; and
actuating the detent to release the discharged staples from the staple exit areas.

11. The method of claim 10 wherein said step of inserting the distal end of the stapler within the vessel includes positioning a wall of the graft adjacent to a wall of the vessel.

12. The method of claim 10 wherein said step of discharging includes advancing an actuator in a forward direction which causes a pusher associated with a staple to move radially outward from a center of the stapler thereby discharging the staples.

13. The method of claim 10 wherein said step of actuating said detent to release the discharged staples includes rotating the detent about a central axis of the stapler thereby causing the arms to release the staples through the staple exit areas.

14. The method of claim 10 wherein the staples are discharged through the staple exit areas simultaneously.

15. The method of claim 10 wherein at least one of the staples is formed from a plurality of opposed loops connected by a central element.

16. The method of claim 15 wherein the staples have a first condition in which the opposed loops are open while stored in the stapler and a second condition in which the opposed loops are at least partially closed when discharged from an associated staple exit area.

17. A surgical stapler system comprising:
a casing with a plurality of staple exit areas; a cartridge within said casing, said cartridge including a plurality of radially mounted pushers with distal ends; a plurality of staples, one of said plurality of staples associated with each of said pushers; an actuating mechanism adapted to advance said pushers along said cartridge to push said staples through said staple exit areas; and a plurality of anvils associated with said cartridge, said anvils having a first position toward said cartridge and a second position away from said cartridge;
a plurality of bolsters, one of each of said plurality of bolsters associated with one of each of said plurality of anvils; and
a graft, said graft disposed between said anvils and said pushers,
wherein said actuating mechanism may be utilized to advance said staples through said staple exit areas when said anvils are in said first position, such that said anvils interfere with said staples to assist said staples into loops capturing said graft, said bolster, and a vessel.

18. The stapler of claim 17 wherein at least one of said staples is made from a memory alloy.

19. The stapler of claim 17 wherein at least one of said staples is formed from a U-shaped section with elongate legs and a central portion connecting the legs.

20. The stapler of claim 19 wherein said at least one staple has a first condition in which said elongate legs are straight while stored in the stapler and a second condition in which said elongate legs are at least partially closed when discharged from an associated staple exit area.

21-50. (canceled)
